(19) United States
(12) Patent Application Publication
(10) Pub. No.: US 2023/0120326 A1
(43) Pub. Date: Apr. 20, 2023

(54) FOOT SANITIZING ASSEMBLY AND METHOD

(71) Applicant: Paula Remedios, Atlanta, GA (US)

(72) Inventor: Paula Remedios, Atlanta, GA (US)

(21) Appl. No.: 17/501,503

(22) Filed: Oct. 14, 2021

(51) Int. Cl. A61L 2/00; B08B 3/08; B08B 3/02; A61L 2/18; A61L 2/24; A61L 2/26

(52) U.S. Cl. CPC A61L 2/0088; B08B 3/08; B08B 3/02; A61L 2/18; A61L 2/24; A61L 2/26; B08B 2203/0223; A61L 2202/15; A61L 2202/14

(57) ABSTRACT

A foot sanitizing assembly includes a housing defined by an outer wall. The outer wall includes a top wall, a bottom wall, and a perimeter wall is attached to and extending between the top and bottom walls. A reservoir is positioned within the housing and contains a fluid. The fluid is a sanitizing agent. A pump is positioned within the housing. A supply conduit is fluidly coupled to the reservoir and to the pump and draws the fluid from the reservoir. A plurality of apertures extending through the top wall. A plurality of dispersant tubes is positioned within the housing. Each of the apertures is fluidly coupled to one of the dispersant tubes and each of the dispersant tubes is fluidly coupled to the pump. The pump receives fluid from the reservoir and ejects the fluid outwardly of the apertures when the pump is turned on.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to foot cleaning devices and more particularly pertains to a new foot cleaning device for assisting a person in cleaning the bottom side of their foot or footwear to prevent the spread of germs across a floor surface.

### BRIEF SUMMARY OF THE INVENTION

An embodiment of the disclosure meets the needs presented above by generally comprising a housing defined by an outer wall. The outer wall includes a top wall, a bottom wall, and a perimeter wall is attached to and extending between the top and bottom walls. A reservoir is positioned within the housing and contains a fluid. The fluid is a sanitizing agent. A pump is positioned within the housing. A supply conduit is fluidly coupled to the reservoir and to the pump and draws the fluid from the reservoir. A plurality of apertures extending through the top wall. A plurality of dispersant tubes is positioned within the housing. Each of the apertures is fluidly coupled to one of the dispersant tubes and each of the dispersant tubes is fluidly coupled to the pump. The pump receives fluid from the reservoir and ejects the fluid outwardly of the apertures when the pump is turned on.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top isometric view of a foot sanitizing assembly and method according to an embodiment of the disclosure.

FIG. 2 is a top view of an embodiment of the disclosure.

FIG. 3 is a cross-sectional view of an embodiment of the disclosure taken along line 3-3 of FIG. 1.

FIG. 4 is a schematic view of an embodiment of the disclosure.

### DETAILED DESCRIPTION OF THE INVENTION

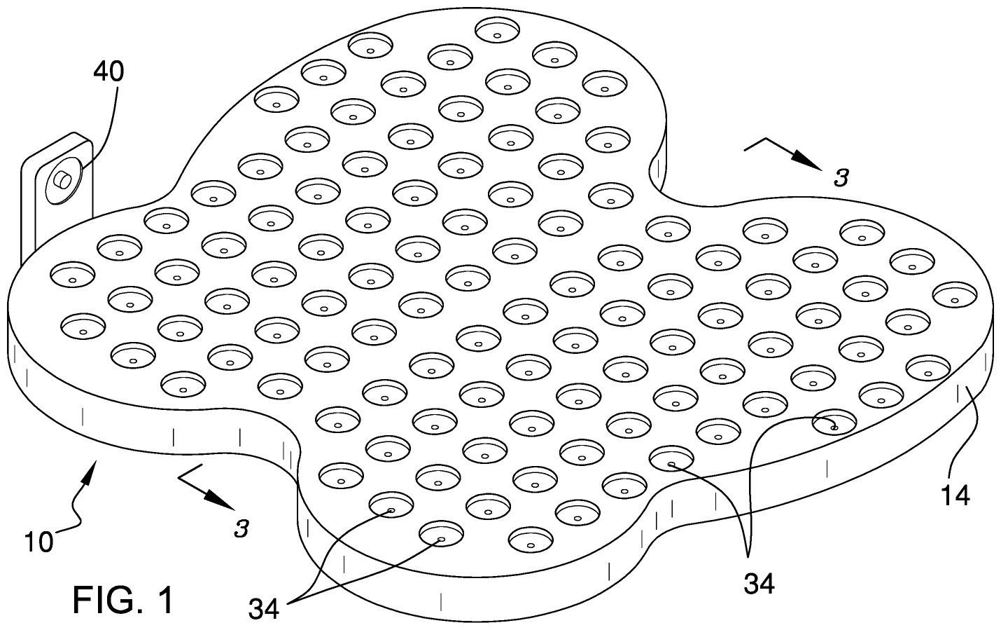

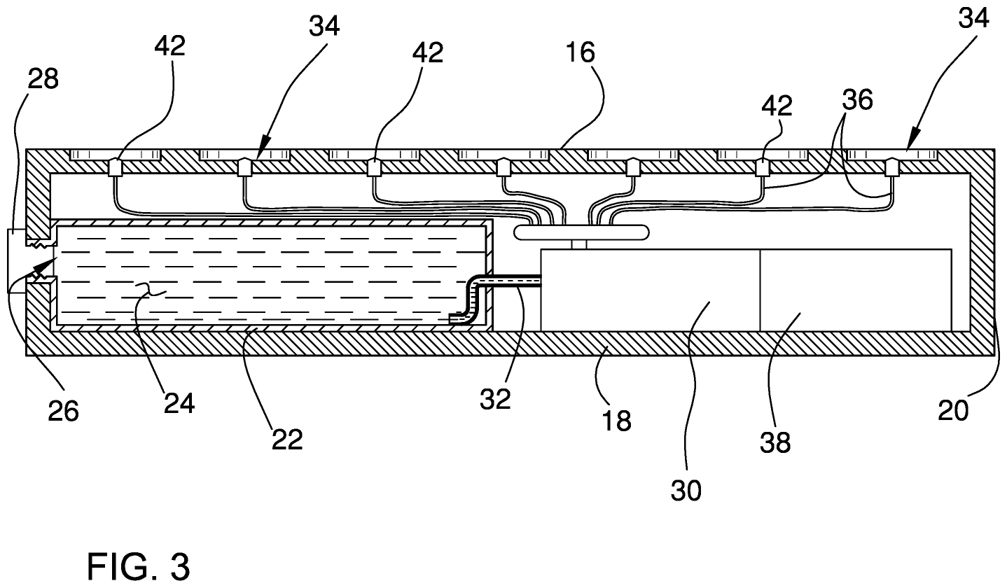

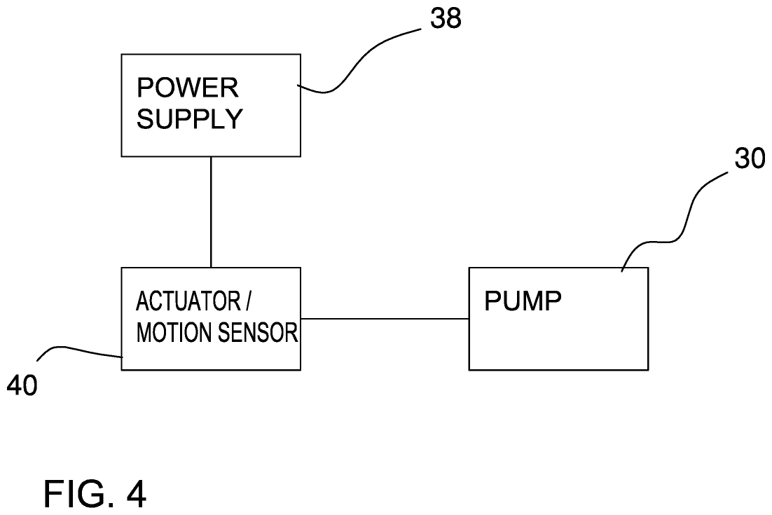

With reference now to the drawings, and in particular to FIGS. 1 through 4 thereof, a new foot cleaning device embodying the principles and concepts of an embodiment of the disclosure and generally designated by the reference numeral 10 will be described.

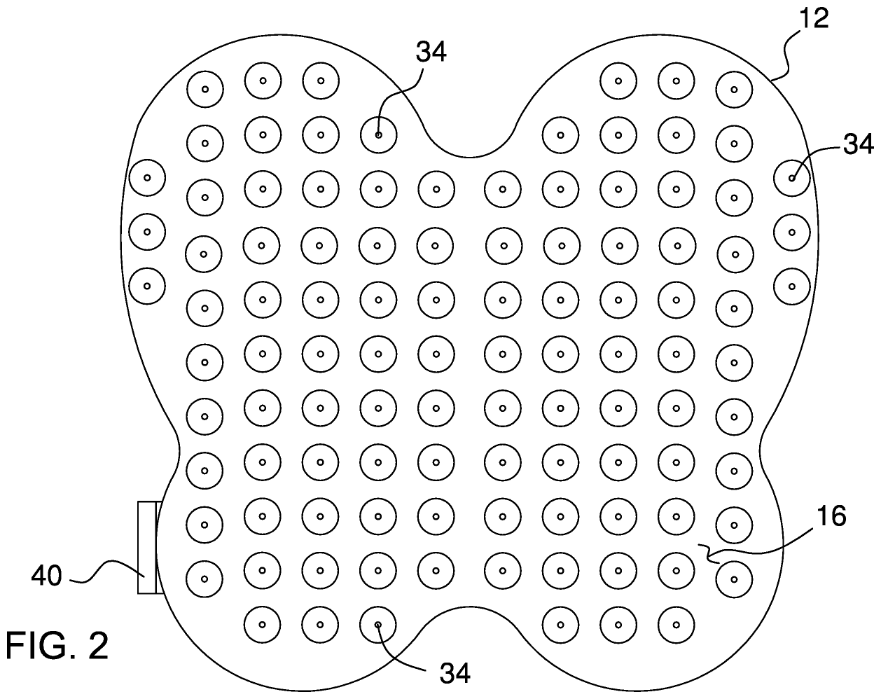

As best illustrated in FIGS. 1 through 4, the foot sanitizing assembly 10 and method generally comprises a housing 12 that is defined by an outer wall 14. The outer wall 14 includes a top wall 16, a bottom wall 18, and a perimeter wall 20 is attached to and extends between the top 16 and bottom 18 walls. The housing 12 has a length and width each being between 6.0 inches and 18.0 inches and a height that is no greater than 3.0 inches. The housing 12 may have any conventional shape including rectangular, round, oval or the like. As can be seen in FIG. 2, the housing may have a shape that is conducive for receiving a pair of feet. The housing 12 may be comprised of materials conventional to personal weight scales such as plastics, metals and the like. The top wall 16 may have an outer surface having a non-slip coating or material such as silicones and elastomeric materials.

A reservoir 22 is positioned within the housing 12. The reservoir 22 is configured to contain a fluid 24. The fluid 24 will typically comprise a conventional a sanitizing agent which is used for killing viruses and bacteria. These may include, for example, alcohol based sanitizing agents or common antibacterial compounds such as benzethonium chloride. A fill opening 26 may be positioned in the outer wall. The fill opening is in fluid communication with the reservoir 22 and a cover 28 is removably positioned on the fill opening.

A pump 30 is positioned within the housing 12 and a supply conduit 32 is fluidly coupled to the reservoir 22 and to the pump 30. The pump 30 is thereafter configured to draw fluid 24 from the reservoir 22. A plurality of apertures 34 extends through the top wall 16. A plurality of dispersant tubes 36 is positioned within the housing 12. Each of the apertures 34 is fluidly coupled to one of the dispersant tubes 36 and each of the dispersant tubes 36 is fluidly coupled to the pump 30. In this manner, when the pump 30 is turned on it receives fluid from the reservoir 22 and pumps the fluid into the dispersant tubes 36 to eject the fluid outwardly of the apertures 34. The pump 30 is powered by a power supply 38 that is electrically coupled to the pump 30. The power supply 38 may comprise a battery mounted within the housing 12 or a power plug electrically coupled to the pump 30 and pluggable into a standard wall outlet.

An actuator 40 is in communication with the pump and turns on the pump 30 when the actuator 40 is actuated. In one embodiment the actuator 40 comprises a motion sensor. The pump 30 may be turned on for a preselected amount of time after the actuator 40 is actuated. This preselected amount of time will typically be less than 10.0 seconds. However, the pump 30 may be constructed such that it is only turned on for a single pulse of fluid 24 dispersant.

While not necessary, a plurality of nozzles 42 may be provided wherein each of the apertures 34 is covered with one of the nozzles 42. The nozzles 42 atomize and spread out the fluid 24 when the fluid 24 is ejected from an associated one of the apertures 34.

In use, the housing 12 is filled with the sanitizing fluid 24 and a person places their foot, or feet, onto the housing 12 such that the actuator detects the foot. This will cause the pump 30 to be turned on and the bottom of the foot to be sprayed with the sanitizing fluid 24. The foot will then be sanitized to prevent the spread of bacteria and viruses across a floor surface.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A foot sanitizing assembly comprising:
a housing being defined by an outer wall, the outer wall including a top wall, a bottom wall, and a perimeter wall being attached to and extending between the top and bottom walls;
a reservoir being positioned within the housing, the reservoir being configured to contain a fluid, the fluid being a sanitizing agent;
a pump being positioned within the housing;
a supply conduit being fluidly coupled to the reservoir and to the pump, the pump being configured to draw fluid from the reservoir;
a plurality of apertures extending through the top wall; and
a plurality of dispersant tubes being positioned within the housing, each of the apertures being fluidly coupled to one of the dispersant tubes, each of the dispersant tubes being fluidly coupled to the pump, wherein the pump receives fluid from the reservoir and ejects the fluid outwardly of the apertures when the pump is turned on.

2. The foot sanitizing assembly according to claim 1, wherein the housing has a length and width each between 6.0 inches and 18.0 inches and a height being no greater than 3.0 inches.

3. The foot sanitizing assembly according to claim 1, further including a power supply being electrically coupled to the pump.

4. The foot sanitizing assembly according to claim 3, further including an actuator being in communication with the pump and turning on the pump when the actuator is actuated.

5. The foot sanitizing assembly according to claim 4, wherein the actuator comprises a motion sensor.

6. The foot sanitizing assembly according to claim 5, wherein the pump is turned on for a preselected amount of time after the actuator is actuated, the preselected amount of time being less than 10.0 seconds.

7. The foot sanitizing assembly according to claim 1, further including a plurality of nozzles, each of the apertures being covered with one of the nozzles, the nozzles atomizing the fluid when the fluid is ejected from an associated one of the apertures.

8. The foot sanitizing assembly according to claim 1, further including a fill opening being positioned in the outer wall and being in fluid communication with the reservoir, a cover being removably positioned on the fill opening.

9. A foot sanitizing assembly comprising:
a housing being defined by an outer wall, the outer wall including a top wall, a bottom wall, and a perimeter wall being attached to and extending between the top and bottom walls, the housing having a length and width each between 6.0 inches and 18.0 inches and a height being no greater than 3.0 inches;
a reservoir being positioned within the housing, the reservoir being configured to contain a fluid, the fluid being a sanitizing agent;
a pump being positioned within the housing;
a supply conduit being fluidly coupled to the reservoir and to the pump, the pump being configured to draw fluid from the reservoir;
a plurality of apertures extending through the top wall;
a plurality of dispersant tubes being positioned within the housing, each of the apertures being fluidly coupled to one of the dispersant tubes, each of the dispersant tubes being fluidly coupled to the pump, wherein the pump receives fluid from the reservoir and ejects the fluid outwardly of the apertures when the pump is turned on;
a power supply being electrically coupled to the pump;
an actuator being in communication with the pump and turning on the pump when the actuator is actuated, the actuator comprising a motion sensor, the pump being turned on for a preselected amount of time after the actuator is actuated, the preselected amount of time being less than 10.0 seconds;
a plurality of nozzles, each of the apertures being covered with one of the nozzles, the nozzles atomizing the fluid when the fluid is ejected from an associated one of the apertures; and
a fill opening being positioned in the outer wall and being in fluid communication with the reservoir, a cover being removably positioned on the fill opening.

10. A method of sanitizing a person's foot comprising the steps of:
positioning a person's foot on a housing being defined by an outer wall, the outer wall including a top wall, a bottom wall, and a perimeter wall being attached to and extending between the top and bottom walls, a reservoir being positioned within the housing, a fluid being positioned within the reservoir, the fluid being a sanitizing agent, a pump being positioned within the housing, a supply conduit being fluidly coupled to the reservoir and to the pump to allow the pump to draw fluid from the reservoir, a plurality of apertures extending through the top wall, a plurality of dispersant tubes being positioned within the housing, each of the apertures being fluidly coupled to one of the dispersant tubes, each of the dispersant tubes being fluidly coupled to the pump
actuating an actuator to turn the pump for a preselected amount of time, the actuator being a motion sensor in communication with the pump, the actuator being mounted on the housing and being actuated when the foot is positioned on the housing; and
ejecting of the fluid from the apertures by the pump when the actuator is actuated such that the fluid is sprayed onto a bottom side of the foot.
